(19) United States
(12) Patent Application Publication
(10) Pub. No.: US 2025/0177211 A1
(43) Pub. Date: Jun. 5, 2025

(54) WEDGELOCK EARPLUG SYSTEMS

(71) Applicant: Wade O'Neill, Ethel, LA (US)

(72) Inventor: Wade O'Neill, Ethel, LA (US)

(21) Appl. No.: 18/526,517

(22) Filed: Dec. 1, 2023

(51) Int. Cl. A61F 11/08

(52) U.S. Cl. CPC A61F 11/085

(57) ABSTRACT

Wedgelock earplug system, designed to be worn and used to protect the user from hearing loss caused by loud environments. Further, the device can be used to effectively ‘soundproof’ the user-wearer from ambient noise so he/she can concentrate. The device preferably has a plastic push pin with a knob that features a middle ring and an outer ring which may be made from polyurethane foam.

### CROSS-REFERENCE TO RELATED APPLICATION

The present application is related to and claims priority from prior provisional application Ser. No. 63/429,635, filed Dec. 2, 2022 which application is incorporated herein by reference.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever. 37 CFR 1.71(d).

### BACKGROUND OF THE INVENTION

The following includes information that may be useful in understanding the present invention(s). It is not an admission that any of the information provided herein is prior art, or material, to the presently described or claimed inventions, or that any publication or document that is specifically or implicitly referenced is prior art.

### 1. Field of the Invention

The present invention relates generally to the field of hearing protection and more specifically relates to ear plugs.

### 2. Description of the Related Art

Many people experience hearing damage that can be caused by loud environments. Hearing damage can lead to significant loss of hearing or even deafness in extreme cases which is highly undesirable. Many earplugs are the pinch and roll design and don't fit properly so are uncomfortable or may fall out during wear. Often the user may have dirty fingers which may be imparted on to the plugs before insertion which is not healthy. It is desirable that hearing protection means be effective above and beyond what is currently available.

Various attempts have been made to solve the above-mentioned problems such as those found in U.S. Pat. No. 7,512,243 to Haussmann. This art is representative of ear plugs. None of the above inventions and patents, taken either singly or in combination, is seen to describe the invention as claimed.

Ideally, a hearing protection means should provide effective ear protection and yet would operate reliably and be manufactured at a modest expense. Thus, a need exists for a reliable ear plug system to avoid the above-mentioned problems.

### BRIEF SUMMARY OF THE INVENTION

In view of the foregoing disadvantages inherent in the known hearing protection art, the present invention provides a novel wedgelock earplug system. The general purpose of the present invention, which will be described subsequently in greater detail is to provide a reliable ear protection means.

The wedgelock earplug system is designed, as disclosed herein, to be worn and used to protect the user from hearing loss caused by loud environments. The device can be used to effectively ‘soundproof’ the user-wearer from ambient noise so he/she can concentrate. The device in preferred embodiments has a plastic push pin with a knob that features a middle ring and an outer ring which may be made from polyurethane foam. The system is designed to protect the hearing of the user-wearer. The device can be adjusted to fit any size ear canal and locked into place to avoid unwanted adjustments. By tight fitting it reduces the chances of the earplugs falling out. The plug is skinnier where it is inserted into the ear canal. It has a flange on the end with a knob. It is designed to fit comfortably and tighter to reduce noise.

In certain embodiments the wedgelock earplug system is designed to be worn and used to protect the user from hearing loss caused by loud environments comprising: a wedgelock earplug assembly having a body with an insertion end as an ear cone; an inner volume; and a distal end comprising a push-pin assembly; and wherein the push-pin assembly has a push pin and is able to move in relation to the ear cone, the push-pin assembly able to incrementally deform the ear cone for creating a user-preferred fit within an ear cavity of a user-wearer designed to protect hearing of the user-wearer. The present invention is adjustable. The devices may comprise Polyethylene, Terephthalate or PET (plastic), Nylon thread, Latex rubber, Silicone. As designed the present invention has an outer diameter that is caused to effectively increase and decrease via mechanical means and can lock into place.

A method of using a wedgelock earplug system is also disclosed herein, comprising the steps of: providing a wedgelock earplug system including: a wedgelock earplug assembly having; a body with an insertion end as an ear cone; an inner volume; and a distal end comprising a push-pin assembly; wherein the push-pin assembly has a push pin and is able to move in relation to the ear cone, the push-pin assembly able to incrementally deform the ear cone for creating a user-preferred fit within an ear cavity of a user-wearer designed to protect hearing of the user-wearer; inserting each the wedgelock earplug assembly within a respective the ear cavity of the user-wearer; and taking up a gap, via manual manipulation, such that the wedgelock earplug assembly is friction-fit to the ear cavity of the user-wearer. The method may further comprise the steps of: pushing the push pin to allow the wedgelock earplug assembly to become uncoupled with the ear cavity of the user-wearer and removed or re-adjusted.

The present invention holds significant improvements and serves as a wedgelock earplug system. For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and method(s) of use for the present invention, wedgelock earplug system, constructed and operative according to the teachings of the present invention.

FIG. 1 shows a side view illustrating a wedgelock earplug of the wedgelock earplug system according to an embodiment of the present invention.

FIG. 2 is a perspective view illustrating the wedgelock earplug according to an embodiment of the present invention of FIG. 1.

FIG. 3 is a perspective view illustrating the earplug according to an embodiment of the present invention of FIG. 1.

FIG. 4 is another view illustrating the earplug according to an embodiment of the present invention of FIG. 1.

FIG. 5 is another view illustrating the earplug according to an embodiment of the present invention of FIG. 1.

The various embodiments of the present invention will hereinafter be described in conjunction with the appended drawings, wherein like designations denote like elements.

### DETAILED DESCRIPTION

As discussed above, embodiments of the present invention relate to an ear protection means and more particularly to a wedgelock earplug system as used to improve the effectiveness of ear protection to avoid hearing loss.

Generally referring now to the wedgelock earplug system; the system is designed to be worn and used, as such, to protect the user from hearing loss caused by loud environments. Further, the device can be used to effectively ‘soundproof’ the user-wearer from ambient noise so he/she can concentrate. The device preferably comprises a plastic push pin with a knob that features a middle ring and an outer ring which may be made from polyurethane foam. The reader should appreciate that other suitably equivalent materials may be used.

During use, when the push pin is retracted, the adjustable outer ring pulls the silicone outer shell. As it retracts, the push pin slides halfway out of the body. The body is a plastic sleeve with a flange on one end and preferably a ¼ hole on the other side so that it can slide in and out smoothly respectively in relation to each other. During the retraction, the middle ring acts as a cap by sealing the body and preventing sound from entering the air-filled cavity of the wedgelock earplug system. The air holes on the back of the body allow air to go into the air cavity when the push pin is fully inserted, thus allowing the wedgelock earplug system to lock into a person's ears.

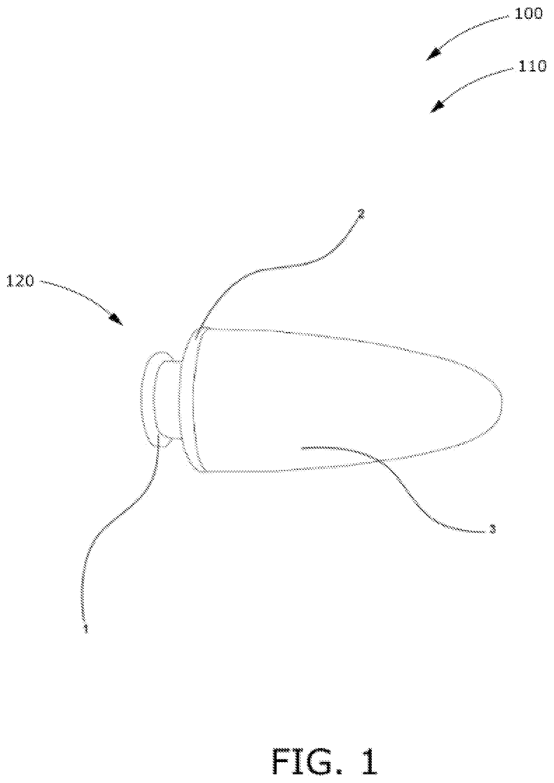

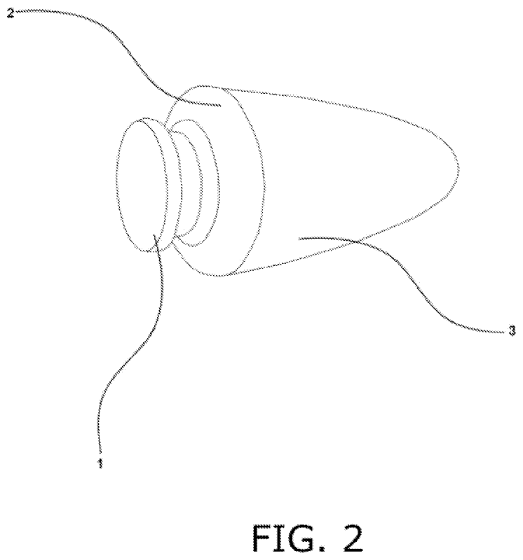

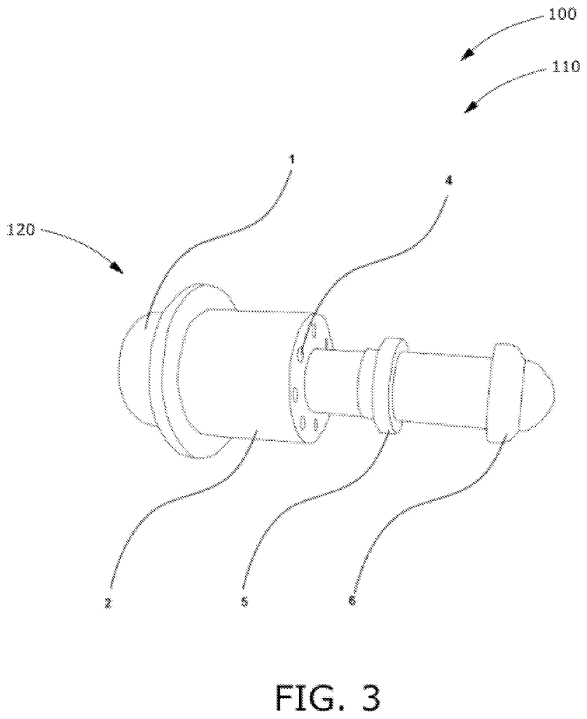

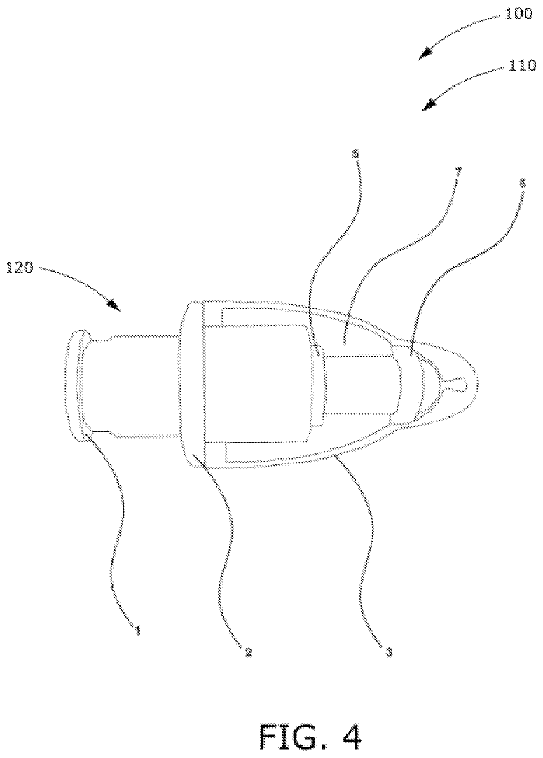

Additionally, the sophistication of the wedgelock earplug system is that it comes with a soft outer ear shell for comfort in use. The entire unit is user-friendly. Relating now to the FIGS. 1-4 the following components are numbered as such: Push pin 1 allows the Ear cone 3 to contract and expand; Body 2 allows the Push pin 1 to slide for full insertion and retraction; Ear Cone 3 is preferably made with silicone that contracts and expand based on the amount of air in the air cavity of the wedgelock earplug system. Seven air holes 4 are preferably located on the inferior of the body 2. This structural feature allows air to go into the air cavity when the push pin 1 is fully inserted, thus allowing the wedgelock earplug system to effectively lock into a person's ears; Middle ring 5 acts as a cap by sealing the body 2 and preventing sound from entering the air-filled cavity of the wedgelock earplug system; the Outer ring 6 is adjustable, it pulls and push the silicone outer shell or the Ear Cone 3; and Air cavities 7, when fully inflated allows the wedgelock earplug system to lock into a person's ears.

The earplug has a preferred diameter of ¼″ at the tip that goes into the ear canal. The tip is soft and malleable for ease of insertion. The plastic body is molded into the silicone. The one end has a flange and the opposing end comprises a ⅛″ hole. The outside of the body has ribs so the silicone will adhere to it. On the inside of the body are a series of channels and ridges. In it there is a shaft that fits snuggly to the body. It also has grooves that correspond with the grooves of the body to ensure locking adjustment into place and release to slide back easily. The shaft also fits through the ⅛″ hole. It slides back and forth through a channel molded into the silicone. The device has a pointed yet rounded off tip. The tip pushes through the tight spot in the channel which causes the ‘skinny end’ to expand. When the shaft is pulled back the tip ‘shrinks’ back up because it is elastic.

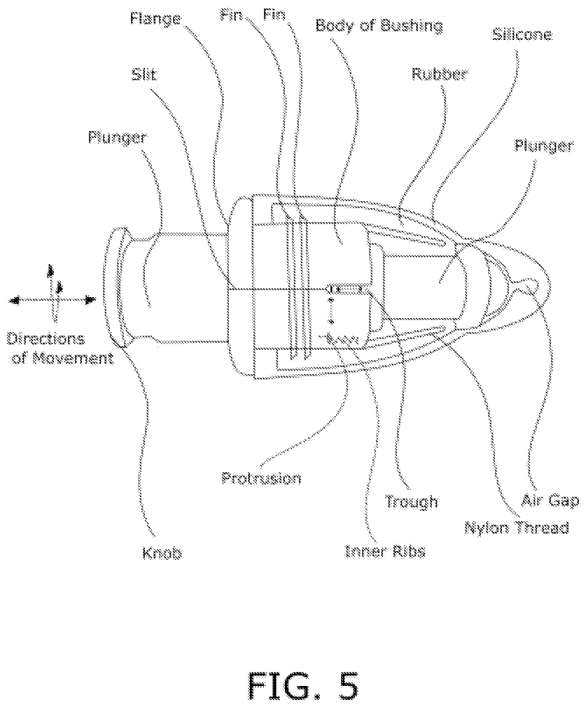

Preferred embodiments may comprise a plastic bushing with a flange and a cylinder with thin walls. It may comprise fins to keep it in position within the silicone body. Further, it may comprise a trough cut out the same width as the protrusion on the plunger. It also has inner ribs formed in the inner diameter of the cylinder. Under normal operation the protrusion rests in the trough. This allows the plunger to move up and down freely. This allows the tip of the plunger to come into contact with the bulb which is merely a narrow gap in the rubber. This causes the rubber outer diameter to effectively increase. Once the adjustment is desired, a twist of the knob will allow the protrusion to move from the trough to the inner ribs thus locking the adjustment in place. A slit is located down the front that allows for flexibility and aids in the ease of initial insertion of the plunger during production. Nylon threads are preferably attached to the bottom of the cylinder. These keep the rubber from stretching longways while allowing the outer diameter to effectively enlarge. (FIG. 5).

The wedgelock earplug system may be sold as a kit having at least one set of user instructions. The kit has instructions such that functional relationships are detailed in relation to the structure of the invention (such that the invention can be used, maintained, or the like in a preferred manner). The kit may be manufactured and provided for sale in a wide variety of sizes and shapes for a wide assortment of applications. Upon reading this specification, it should be appreciated that, under appropriate circumstances, considering such issues as design preference, user preferences, marketing preferences, cost, structural requirements, available materials, technological advances, etc., other kit contents or arrangements such as, for example, including more or less components, customized parts, different color combinations, parts may be sold separately, etc., may be sufficient.

Referring now using numerals of reference to FIGS. 1-5, a wedgelock earplug system 100 is shown designed to be worn and used to protect the user from hearing loss caused by loud environments, the present invention comprising: a wedgelock earplug assembly 110 having a body 2 with an insertion end as an ear cone 3; an inner volume; and a distal end comprising a push-pin assembly 120; wherein the push-pin assembly 120 has a push pin 1 and is able to move in relation to the ear cone 3; the push-pin assembly 120 able to incrementally deform the ear cone 3 for creating a user-preferred fit within an ear cavity of a user-wearer designed to protect the hearing of the user-wearer.

The push-pin assembly 120 is able to be manually manipulated by the user-wearer to determine an amount that the ear cone 3 is able to incrementally deform. The ear cone 3 is able to be contracted and expanded in relation to the ear cavity of a user-wearer as desired by push and pull movements; wherein the ear cone 3 preferably comprises silicone. The ear cone 3 may comprise alternate materials that are suitably equivalent.

In certain embodiments the body 2 of the wedgelock earplug system 100 further comprises air holes 4 such that the ear cone 3 is able to be contracted and expanded in relation to a volume of air in the ear cavity; wherein the air holes 4 comprise at least two of the air holes 4 and preferably exactly seven of the air holes 4. The air holes 4 permit air to go into an air cavity when the push pin 1 is fully inserted, thus permitting the wedgelock earplug assembly 110 to lock into the ear cavity of the user-wearer. The wedgelock earplug assembly 110 of the wedgelock earplug system 100 may further comprise a middle ring 5 and an outer ring 6; wherein the middle ring 5 is located relatively adjacent the outer ring 6. The push pin 1 is preferably oriented in series respectively next to the middle ring 5 and then the outer ring 6, the outer ring 6 adjacent to the ear cavity of the user-wearer when in an in-use condition; wherein the in-use condition comprises a position wherein the ear cone 3 is suitably compressed to the ear cavity of the user-wearer. The in-use condition comprises a comfortable friction-fit; wherein the friction-fit is designed to reduce occurrences of the wedgelock earplug assembly 110 losing contact-coupling from the ear cavity of the user-wearer.

A wedgelock earplug system 100 to be worn and used to protect the user from hearing loss caused by loud environments in a preferred embodiment comprises: a wedgelock earplug assembly 110 having a body 2 with an insertion end as an ear cone 3; an inner volume; and a distal end comprising a push-pin assembly 120; wherein the push-pin assembly 120 has a push pin 1 and is able to move in relation to the ear cone 3; the push-pin assembly 120 able to incrementally deform the ear cone 3 for creating a user-preferred fit within an ear cavity of a user-wearer designed to protect hearing of the user-wearer.

The push-pin assembly 120 is able to be manually manipulated by the user-wearer to determine an amount that the ear cone 3 is able to the incrementally deform; wherein the ear cone 3 is able to be contracted and expanded in relation to the ear cavity of a user-wearer as desired by push and pull sliding movements; wherein the ear cone 3 preferably comprises silicone.

The body 2 further comprises air holes 4; wherein the ear cone 3 is able to be contracted and expanded in relation to a volume of air within the ear cavity. The air holes 4 comprise exactly seven of the air holes 4 in preferred embodiments. The air holes 4 allow air to go into an air cavity when the push pin 1 is fully inserted, thus permitting the wedgelock earplug assembly 110 to substantially lock into the ear cavity of the user-wearer.

The wedgelock earplug assembly 110 may further comprise a middle ring 5 and an outer ring 6; wherein the middle ring 5 is located relatively adjacent the outer ring 6; and the push pin 1 is oriented in series respectively next to the middle ring 5 and then the outer ring 6, the outer ring 6 adjacent to the ear cavity of the user-wearer when in an in-use condition. The in-use condition comprises a position wherein the ear cone 3 is suitably compressed next to and against the ear cavity of the user-wearer; wherein the in-use condition comprises a comfortable friction-fit; and wherein the friction-fit is designed to reduce occurrences of the wedgelock earplug assembly 110 losing contact-coupling from the ear cavity of the user-wearer.

FIG. 5 comprises an adjustable ear plug with a plastic bushing. Further it preferably comprises a flange and a cylinder with thin walls. The present invention has fins to keep it in place within the silicone body. The device has a trough cut out the same width as the protrusion on the plunger. It also has inner ribs formed in the inner diameter of the cylinder. Under normal operation the protrusions rest in the trough. This allows the plunger to come into contact with the bulb which is merely a narrow gap in the rubber. This causes the rubber outer diameter to effectively increase. Once the adjustment desired is reached a twist of the knob allows the protrusion to move from the trough to the inner ribs thus locking the adjustment in place. The slit down the front allows for flexibility and aids in the initial insertion of the plunger during production. Nylon threads are attached to the bottom of the cylinder. These keep the rubber from stretching long ways while allowing the outer diameter to enlarge.

A method of using a wedgelock earplug system 100 is also disclosed herein, comprising the steps of: providing a wedgelock earplug system 100 including: a wedgelock earplug assembly 110 having a body 2 with an insertion end as an ear cone 3; an inner volume; and a distal end comprising a push-pin assembly 120; wherein the push-pin assembly 120 has a push pin 1 and is able to move in relation to the ear cone 3, the push-pin assembly 120 able to incrementally deform the ear cone 3 for creating a user-preferred fit within an ear cavity of a user-wearer designed to protect hearing of the user-wearer; inserting each wedgelock earplug assembly 110 within a respective the ear cavity of the user-wearer; and taking up a gap, via manual manipulation, such that the wedgelock earplug assembly 110 is friction-fit to the ear cavity of the user-wearer. The method may further comprise the steps of: pushing the push pin 1 to allow the wedgelock earplug assembly 110 to become uncoupled with the ear cavity of the user-wearer and removed or re-adjusted.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. A wedgelock earplug system to be worn and used to protect the user from hearing loss caused by loud environments comprising:
a wedgelock earplug assembly having;
a body with
an insertion end as an ear cone;
an inner volume; and
a distal end comprising a push-pin assembly; and
wherein said push-pin assembly has a push pin and is able to move in relation to said ear cone, said push-pin assembly able to incrementally deform said ear cone for creating a user-preferred fit within an ear cavity of a user-wearer designed to protect hearing of the user-wearer.

2. The wedgelock earplug system of claim 1 wherein said push-pin assembly is able to be manually manipulated by said user-wearer to determine an amount that said ear cone is able to said incrementally deform.

3. The wedgelock earplug system of claim 2 wherein said ear cone is able to be contracted and expanded in relation to said ear cavity of said user-wearer as desired by push and pull movements.

4. The wedgelock earplug system of claim 3 wherein said ear cone comprises silicone.

5. The wedgelock earplug system of claim 4 wherein said body further comprises air holes.

6. The wedgelock earplug system of claim 5 wherein said ear cone is able to be contracted and expanded in relation to a volume of air in said ear cavity.

7. The wedgelock earplug system of claim 5 wherein said air holes comprises at least two of said air holes.

8. The wedgelock earplug system of claim 6 wherein said air holes comprises exactly seven of said air holes.

9. The wedgelock earplug system of claim 1 wherein said air holes allow air to go into an air cavity when said push pin is fully inserted, thus permitting said wedgelock earplug assembly to lock into said ear cavity of said user-wearer.

10. The wedgelock earplug system of claim 1 wherein said wedgelock earplug assembly further comprises a middle ring.

11. The wedgelock earplug system of claim 10 wherein said wedgelock earplug assembly further comprises an outer ring.

12. The wedgelock earplug system of claim 11 wherein said middle ring is located relatively adjacent to said outer ring.

13. The wedgelock earplug system of claim 1 wherein said push pin is oriented in series respectively next to said middle ring and then said outer ring, said outer ring adjacent to said ear cavity of said user-wearer when in an in-use condition.

14. The wedgelock earplug system of claim 13 wherein said in-use condition comprises a position wherein said ear cone is suitably compressed to said ear cavity of said user-wearer.

15. The wedgelock earplug system of claim 14 wherein said in-use condition comprises a comfortable friction-fit.

16. The wedgelock earplug system of claim 1 wherein said friction-fit is designed to reduce occurrences of said wedgelock earplug assembly losing contact-coupling from said ear cavity of said user-wearer.

17. A wedgelock earplug system to be worn and used to protect the user from hearing loss caused by loud environments comprising:
a wedgelock earplug assembly having;
a body with
an insertion end as an ear cone;
an inner volume; and
a distal end comprising a push-pin assembly; and
wherein said push-pin assembly has a push pin and is able to move in relation to said ear cone, said push-pin assembly able to incrementally deform said ear cone for creating a user-preferred fit within an ear cavity of a user-wearer designed to protect hearing of the user-wearer;
wherein said push-pin assembly is able to be manually manipulated by said user-wearer to determine an amount that said ear cone is able to said incrementally deform;
wherein said ear cone is able to be contracted and expanded in relation to said ear cavity of a user-wearer as desired by push and pull sliding movements;
wherein said ear cone comprises silicone;
wherein said body further comprises air holes;
wherein said ear cone is able to be contracted and expanded in relation to a volume of air within said ear cavity;
wherein said air holes comprises exactly seven of said air holes;
wherein said air holes allow air to go into an air cavity when said push pin is fully inserted, thus permitting said wedgelock earplug assembly to substantially lock into said ear cavity of said user-wearer;
wherein said wedgelock earplug assembly further comprises a middle ring and an outer ring;
wherein said middle ring is located relatively adjacent said outer ring;
wherein said push pin is oriented in series respectively next to said middle ring and then said outer ring, said outer ring adjacent to said ear cavity of said user-wearer when in an in-use condition;
wherein said in-use condition comprises a position wherein said ear cone is suitably compressed next to and against said ear cavity of said user-wearer;
wherein said in-use condition comprises a comfortable friction-fit; and
wherein said friction-fit is designed to reduce occurrences of said wedgelock earplug assembly losing contact-coupling from said ear cavity of said user-wearer.

18. The wedgelock earplug system of claim 17 further comprising a kit including: at least a pair of said wedgelock earplug assemblies, a carrying case, and a set of user instructions.

19. A method of using a wedgelock earplug system comprising the steps of:
providing a wedgelock earplug system including:
a wedgelock earplug assembly having;
a body with
an insertion end as an ear cone;
an inner volume; and
a distal end comprising a push-pin assembly; and
wherein said push-pin assembly has a push pin and is able to move in relation to said ear cone, said push-pin assembly able to incrementally deform said ear cone for creating a user-preferred fit within an ear cavity of a user-wearer designed to protect hearing of the user-wearer;
inserting each said wedgelock earplug assembly within a respective said ear cavity of said user-wearer; and
taking up a gap, via manual manipulation, such that said wedgelock earplug assembly is friction-fit to said ear cavity of said user-wearer.

20. The method of claim 19 further comprising the steps of: pushing said push pin to allow said wedgelock earplug assembly to become uncoupled with said ear cavity of said user-wearer and removed or re-adjusted.
